(19) United States
(12) Patent Application Publication
(10) Pub. No.: US 2024/0342337 A1
(43) Pub. Date: Oct. 17, 2024

(54) GAS TREATMENT DEVICE AND GAS TREATMENT METHOD

(71) Applicant: CANON KABUSHIKI KAISHA, Tokyo (JP)

(72) Inventors: YUICHI KIKUCHI, Shizuoka (JP); MASATSUGU HONGO, Shizuoka (JP); ATSUSHI UEMATSU, Shizuoka (JP); MASAKI OZAWA, Shizuoka (JP); TAKUMI FURUKAWA, Shizuoka (JP); KAZUHIRO YAMAUCHI, Shizuoka (JP)

(21) Appl. No.: 18/751,449

(22) Filed: Jun. 24, 2024

Related U.S. Application Data
(63) Continuation of application No. PCT/JP2022/048043, filed on Dec. 26, 2022.

(30) Foreign Application Priority Data
Dec. 28, 2021 (JP) 2021-215344
Dec. 21, 2022 (JP) 2022-203896

(51) Int. Cl. A61L 9/22; A61L 9/20; H05H 1/24

(52) U.S. Cl. CPC A61L 9/22; A61L 9/20; H05H 1/245; A61L 2209/12; H05H 2245/10

(57) ABSTRACT

This gas treatment device is provided with a cylindrical casing having a first opening and a second opening on the opposite side from the first opening, a specific plasma actuator disposed in the casing, and an ozone decomposition device, wherein the plasma actuator is disposed so that the direction in which the induced current is emitted is oriented towards the second opening, and produces, by means of the induced current, an airflow in the casing such that the airflow is directed from the first opening towards the second opening, the ozone decomposition device causes ozone included in the airflow to decompose, generating active oxygen in the airflow and causing the airflow to contain the active oxygen, and a gas flowing in from the first opening is treated by the active oxygen.

### CROSS-REFERENCE TO RELATED APPLICATION

This is a continuation of International Application No. PCT/JP2022/048043, filed on Dec. 26, 2022, and designated the U.S., and claims priority from Japanese Patent Application No. 2021-215344 filed on Dec. 28, 2021 and Japanese Patent Application No. 2022-203896 filed on Dec. 21, 2022, the entire contents of which are incorporated herein by reference.

### BACKGROUND OF THE INVENTION

### Field of the Invention

The present disclosure relates to a gas treatment device and a gas treatment method using active oxygen.

### Description of the Related Art

Japanese Patent Application Laid-open No. H06-335518 discloses a nascent oxygen generation device including: an apparatus body that is installed in a place where air flows, formed in a cylindrical shape in which a part of the air passes through, and has an inner surface made of metal with high ultraviolet reflectance; an ultraviolet lamp that is disposed on a shaft inside of the apparatus body and irradiates ultraviolet rays to decompose ozone; and an ozone generator that is provided on the upstream side of an airflow inside of the device body and converts oxygen in the air, which has been introduced into the body, into ozone by discharge. In paragraph of Japanese Patent Application Laid-open No. H06-335518, it is described that ozone generation performance and ozone decomposition performance are improved according to the nascent oxygen generation device, whereby large amounts of nascent oxygen from ozone are generated, and the generated nascent oxygen spreads into a refrigerator to oxidize and decompose bad-smelling substances inside of the refrigerator to deodorize the same.

### SUMMARY OF THE INVENTION

In the nascent oxygen generation device according to Japanese Patent Application Laid-open No. H06-335518, air introduced into the device from the outside comes into contact with active oxygen. Therefore, it is presumed that the nascent oxygen generation device could be used for treatment such as deodorization and sterilization of air outside of the device. Therefore, the present inventors considered applying the nascent oxygen generation device according to Japanese Patent Application Laid-open No. H06-335518 to the treatment of gas. However, the performance of deodorization or sterilization of gas by the nascent oxygen generation device is restrictive.

At least an aspect of the present disclosure is aimed at providing a gas treatment device capable of more effectively treating gas.

Further, at least an aspect of the present disclosure is aimed at providing a gas treatment method capable of more effectively treating gas.

At least an aspect of the present disclosure provides a gas treatment device comprising:
a cylindrical housing having a first opening and a second opening on a side opposite to the first opening;
a plasma actuator arranged inside of the housing; and
an ozone decomposition device, wherein
the plasma actuator comprises a first electrode, a dielectric, and a second electrode,
the dielectric is interposed between the first electrode and the second electrode,
the first electrode and the second electrode are electrically insulated from each other,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the plasma actuator is arranged so that the first direction representing a blowing-out direction of the induced flow is oriented toward the second opening, and causes an airflow containing gas flowing in from the first opening and oriented from the first opening toward the second opening to be generated inside of the housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

Further, at least an aspect of the present disclosure provides a gas treatment device comprising:
a cylindrical housing having a first opening and a second opening on a side opposite to the first opening; and
an ozone decomposition device, wherein
the cylindrical housing comprises a dielectric,
a first electrode representing an exposed electrode provided to coat a part of an inner surface is arranged on the inner surface of the cylindrical housing, and a second electrode electrically insulated from the first electrode via the dielectric is arranged outside of the inner surface of the housing in a cross section of the cylindrical housing along an axial direction,
when a voltage is applied between the first electrode and the second electrode, the gas treatment device generates a dielectric barrier discharge oriented from the first electrode toward the second electrode, and blows out an induced flow containing ozone toward a direction of the second opening representing one direction along the inner surface of the housing from the first electrode, and
the gas treatment device causes an airflow containing gas flowing in from the first opening and oriented from the first opening toward the second opening to be generated inside of the cylindrical housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

Furthermore, at least an aspect of the present disclosure provides a gas treatment method using active oxygen, the method comprising:
a step of preparing a gas treatment device, wherein
the gas treatment device comprises
   a cylindrical housing having a first opening and a second opening on a side opposite to the first opening,
   a plasma actuator arranged inside of the housing, and
   an ozone decomposition device,
the plasma actuator comprises a first electrode, a dielectric, and a second electrode,
the dielectric is interposed between the first electrode and the second electrode,
the first electrode and the second electrode are electrically insulated from each other,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the plasma actuator is arranged so that the first direction representing a blowing-out direction of the induced flow is oriented toward the second opening, and causes an airflow containing gas flowing in from the first opening and oriented from the first opening toward the second opening to be generated inside of the housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

At least an aspect of the present disclosure enables the provision of a gas treatment device capable of more effectively treating gas. Further, at least an aspect of the present disclosure enables the provision of a gas treatment method capable of more effectively treating gas. Further features of the present invention will become apparent from the following description of exemplary embodiments with reference to the attached drawings.

### BRIEF DESCRIPTION OF THE DRAWINGS

FIG. 1 is a schematic perspective view showing the appearance of a gas treatment device according to an aspect of the present disclosure;

FIGS. 2A to 2F are explanatory views of the gas treatment device according to an aspect of the present disclosure;

FIG. 3 is an explanatory view of the arrangement position of a plasma actuator according to an aspect of the present disclosure;

FIGS. 4A to 4C are explanatory views of a gas treatment device according to another aspect of the present disclosure;

FIG. 5 is a cross-sectional schematic view of an evaluation device of a gas treatment device according to the present disclosure;

FIG. 6 is a schematic view of a configuration in a case where the sterilization rate of the gas treatment device according to the present disclosure is evaluated;

FIGS. 7A and 7B are cross-sectional schematic views of a gas treatment device according to Example 3;

FIGS. 8A to 8C are cross-sectional schematic views of a gas treatment device according to Example 4;

FIGS. 9A to 9C are cross-sectional schematic views of a gas treatment device according to Example 5; and

FIGS. 10A to 10C are cross-sectional schematic views of a gas treatment device according to Example 7.

### DESCRIPTION OF THE EMBODIMENTS

In the present disclosure, statements of “from XX to YY” and “XX to YY” each representing a numerical value range mean numerical value ranges including lower limits and upper limits, which are endpoints, unless otherwise particularly specified. When numerical value ranges are stepwise stated, the upper and lower limits of the individual numerical value ranges can optionally be combined. In addition, in the present disclosure, such a statement as, e.g., “at least one selected from the group consisting of XX, YY, and ZZ” means any of XX, YY, ZZ, a combination of XX and YY, a combination of XX and ZZ, a combination of YY and ZZ, and a combination of XX, YY, and ZZ.

Further, in the present disclosure, the “treatment” of an object to be treated using active oxygen includes any treatment achievable by active oxygen, such as surface modification (hydrophilic treatment), sterilization, deodorization, and bleaching of the surface to be treated of the object using active oxygen.

Moreover, “funguses” as targets for “sterilization” according to the present disclosure represent microorganisms, and the microorganisms include, in addition to true funguses, bacteria, single-celled algae, viruses, protozoans, or the like, animal or plant cells (including stem cells, dedifferentiated cells, and differentiated cells), tissue cultures, fused cells (including hybridoma) obtained by gene engineering, dedifferentiated cells, and transformants (microorganisms). Examples of viruses include noroviruses, rotaviruses, influenza viruses, adenoviruses, coronaviruses, measles viruses, rubella viruses, hepatitis viruses, herpes viruses, HIV, or the like. Further, examples of bacteria include Staphylococcus bacteria, E. coli bacteria, Salmonella, Pseudomonas aeruginosa, cholera bacteria, dysentery bacilli, Bacillus anthracis, tubercle bacilli, botulinum, tetanus bacilli, Streptococcus, or the like. Moreover, examples of true funguses include Trichophyton, Aspergillus, Candida, or the like. Accordingly, the “sterilization” according to the present disclosure includes, for example, the deactivation of viruses.

Moreover, active oxygen in the present disclosure includes, for example, free radicals such as superoxide (·O₂⁻) and hydroxy radicals (·OH) produced by the decomposition of ozone (O₃).

Hereinafter, some embodiments of a gas treatment device according to the present disclosure will be specifically illustrated with reference to the drawings. However, the dimensions, materials, shapes, their relative arrangements, or the like of constituting components described in the embodiments shall be appropriately modified depending on the configurations or various conditions of members to which the disclosure is applied. That is, the gas treatment device according to the present disclosure is not limited only to configurations realized in respective aspects.

Further, configurations having the same functions will be denoted by the same reference numbers in the drawings, and their descriptions will be omitted depending on circumstances below.

According to studies by the present inventors, it is presumed that one reason for a restrictive gas treatment effect obtained by a nascent oxygen generation device according to Japanese Patent Application Laid-open No. H06-335518 is as follows.

That is, it is presumed that active oxygen is highly unstable, extremely short with a half-life of 10⁻⁶ seconds for ·O₂⁻ and a half-life of 10⁻⁹ seconds for. OH, and promptly converted into stable oxygen and water.

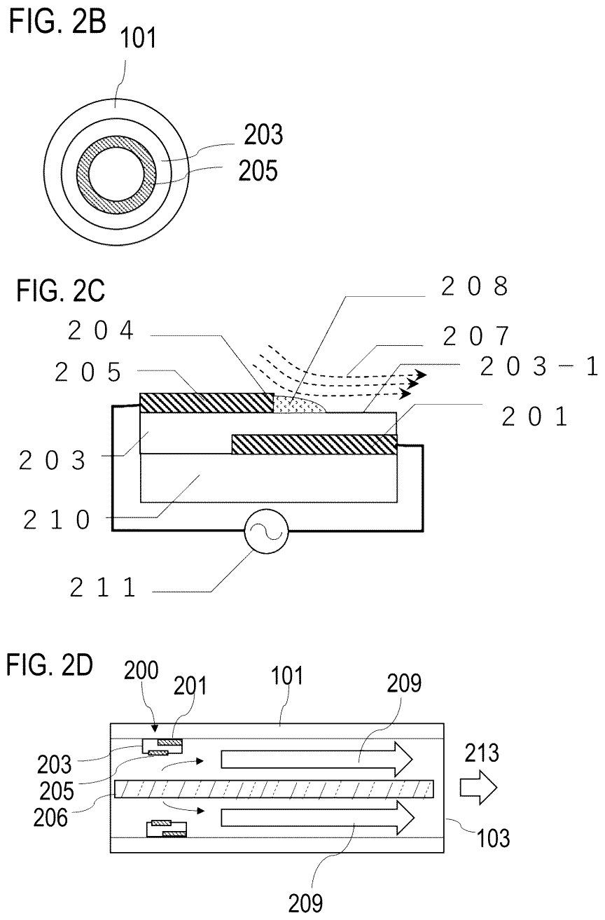

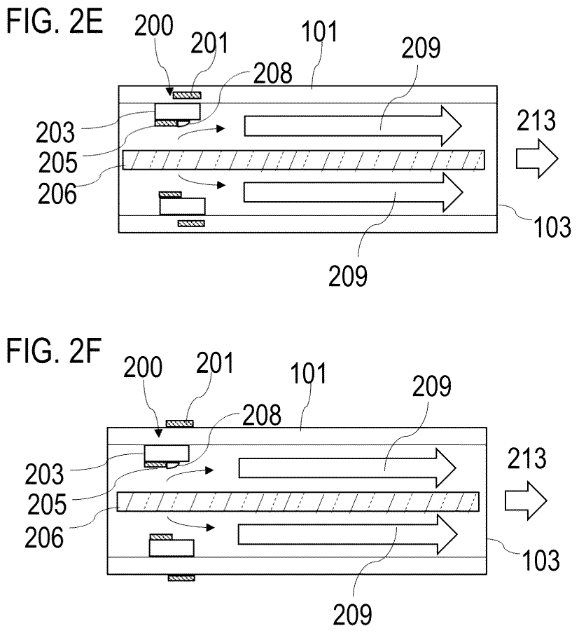

Particularly, the nascent oxygen generation device according to Japanese Patent Application Laid-open No. H06-335518 is installed at a place where an airflow is generated, and a part of air passes through the inside of a cylindrical device body. Specifically, it is disclosed in FIG. 2 of Japanese Patent Application Laid-open No. H06-335518 that an airflow is generated by a refrigerating machine fan 4 installed inside of a refrigerator. Even where active oxygen is generated inside of the device body in such a situation, air flowing in from the outside forms turbulence inside of the device body, and the active oxygen collides with the inner wall of the device body due to the turbulence. As a result, it is presumed that the active oxygen is converted into oxygen and water in an extremely short period of time. Therefore, it is presumed that a contact opportunity between the active oxygen and treatment targets (such as odor substances and bacteria) inside of the device body is extremely restrictive.

By such considerations, the present inventors have made further studies for the purpose of achieving a gas treatment device capable of more reliably using active oxygen for gas treatment to more effectively treat gas. As a result, the present inventors have found that the above gas treatment device and the gas treatment method (that will be called the “gas treatment device or the like” depending on circumstances) contribute to the achievement of the object. Hereinafter, specific aspects of the gas treatment device or the like according to the present disclosure will be described. Note that the gas treatment device or the like according to the present disclosure will not be limited to the specific aspects below.

### First Aspect

A gas treatment device according to a first aspect comprises: a cylindrical housing having a first opening and a second opening on the side opposite to the first opening; a plasma actuator arranged inside of the housing; and an ozone decomposition device.

The plasma actuator comprises a first electrode, a dielectric, and a second electrode laminated together in this order.

The dielectric is interposed between the first electrode and the second electrode, whereby the first electrode and the second electrode are electrically insulated from each other.

Further, the first electrode is an exposed electrode provided on a first surface representing one surface of the dielectric. When a voltage is applied between the first electrode and the second electrode, the plasma actuator generates a dielectric barrier discharge oriented from the first electrode toward the second electrode, and blows out an induced flow containing ozone in a first direction representing one direction along the surface of the dielectric from the first electrode. Further, the plasma actuator is arranged so that the first direction representing the blowing-out direction of the induced flow is oriented to the second opening, and causes an airflow oriented from the first opening toward the second opening to be generated inside of the housing by the induced flow.

Further, the ozone decomposition device decomposes the ozone contained in the airflow generated inside of the housing to generate active oxygen in the airflow. As a result, the active oxygen is contained in the airflow inside of the housing.

The gas treatment device according to this aspect will be described in further detail using the drawings.

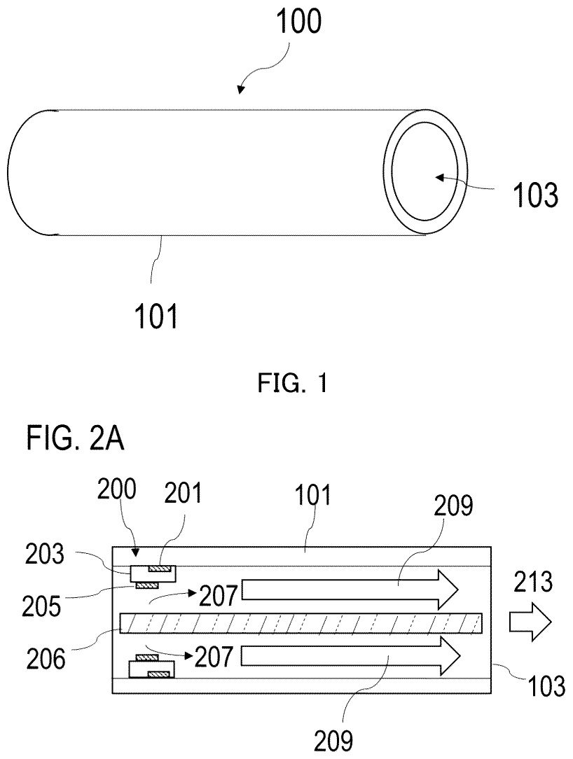

FIG. 1 is a perspective view showing the appearance of a gas treatment device 100 according to this aspect, wherein a cylindrical housing 101 has a first opening (not shown) at one end and a second opening 103 at the opposite end.

FIG. 2A is a cross-sectional view of the gas treatment device 100 along a direction (that will also be called a “longitudinal direction” below) oriented from the first opening (not shown) toward the second opening 103. On the inner surface of the cylindrical housing 101, a plasma actuator 200 is arranged. The plasma actuator 200 comprises a first electrode 205 that is an exposed electrode provided on one surface of a dielectric 203 and a second electrode 201 that is electrically insulated from the first electrode 205 via the dielectric 203.

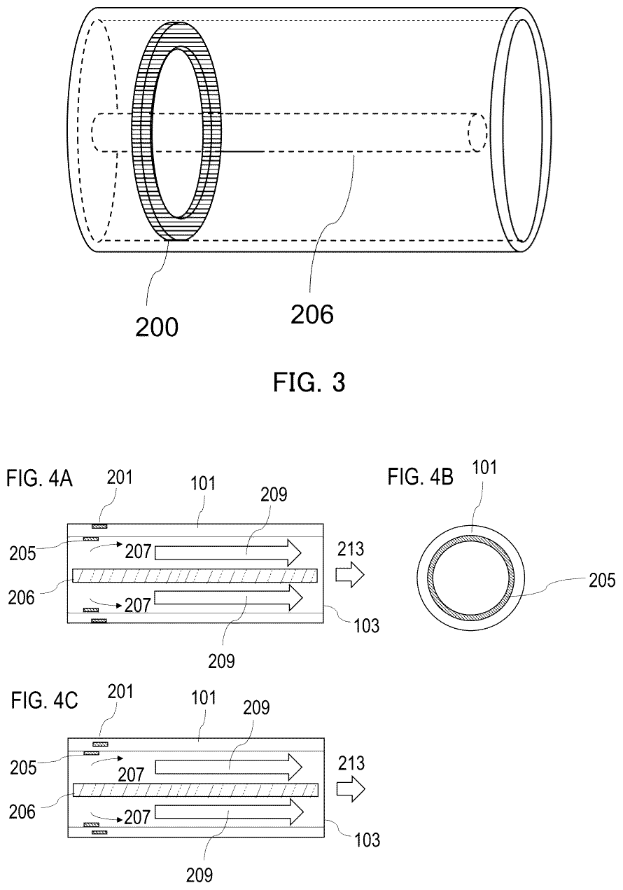

As shown in FIG. 2B viewed from the side of the second opening of the gas treatment device 100 and FIG. 3, which is a perspective view of the gas treatment device 100, the plasma actuator 200 is preferably arranged over the entire circumference of the inner surface of the cylindrical housing 101 in the peripheral direction.

Then, when a voltage is applied between the first electrode 205 and the second electrode 201, the plasma actuator 200 generates a dielectric barrier discharge oriented from the first electrode toward the second electrode, and blows out an induced flow 207 containing ozone in a first direction representing one direction along the surface of the dielectric from the first electrode.

Further, the plasma actuator 200 is arranged so that the first direction representing the blowing-out direction of the induced flow 207 containing the ozone is oriented toward the second opening 103. Then, an airflow 209 in a direction indicated by arrows is generated inside of the cylindrical housing 101 by the induced flow 207 containing the ozone, and air outside of the gas treatment device is taken into the cylindrical housing 101 from the first opening. That is, an airflow containing the gas flowing in from the first opening and oriented from the first opening toward the second opening 103 is generated inside of the housing by the induced flow 207 containing the ozone.

Further, in the cylindrical housing 101, an ultraviolet light source 206 serving as an ozone decomposition device is arranged at the center of the housing along the longitudinal direction. The ultraviolet light source 206 irradiates the induced low with ultraviolet rays to decompose the ozone in the induced flow and generate active oxygen inside of the airflow 209. As a result, the airflow 209 inside of the housing results in an airflow containing the active oxygen, and the gas flowing in from the first opening is treated by the active oxygen. Further, the airflow containing the active oxygen is also capable of treating gas as an object to be treated, which is present in the outflow direction of the airflow containing the active oxygen, with the active oxygen.

Similarly, in a case where the ozone decomposition device 206 is, for example, a heating device or a humidifying device, it is also possible to decompose the ozone in the airflow 209 by heat or water and supply an airflow 213 containing the active oxygen.

According to studies by the present inventors, it is presumed that the active oxygen contained in the airflow 209 generated inside of the housing due to the induced flow from the plasma actuator is capable of maintaining its active state for a longer period of time than a general life (a half-life of 10⁻⁶ seconds for ·O₂⁻ and a half-life of 10⁻⁹ seconds for ·OH) of active oxygen. A reason why the active oxygen generated in the airflow is capable of maintaining the active state for a long period of time is as follows. That is, the airflow 209 generated inside of the housing due to the induced flow representing a unidirectional jet flow is an extremely straightened flow. Therefore, it is assumed that the active oxygen is protected inside of the airflow 209, and its deactivation due to contact with the inner wall or the like of the housing is suppressed. Accordingly, the gas treatment device of the present disclosure is capable of effectively using the generated active oxygen for gas treatment.

On the other hand, it is assumed that air forcibly introduced into the housing by a fan or the like arranged outside of the device forms turbulence inside of the housing and is highly likely to come into contact with the inner wall or the like of the housing, thus making it more likely for the active oxygen generated inside of the housing to be deactivated. Accordingly, the gas treatment device according to the present disclosure preferably does not include other airflow generation units (such as a blowing fan) for generating an airflow other than the airflow 209 generated due to the induced flow inside of the housing.

Furthermore, the plasma actuator 200 preferably has a shape conforming to the inner surface of the cylindrical housing 101. When the cylindrical housing 101 is viewed from the second opening, the plasma actuator 200 may be provided alone at one spot of the inner surface of the cylindrical housing 101 in the peripheral direction, or may be provided plurally at one spot of the inner surface of the cylindrical housing 101 in the peripheral direction. For example, when the cylindrical housing 101 is viewed from the second opening, the ratio of the length at which the plasma actuator 200 is provided to the length of the entire circumference of the inner surface of the cylindrical housing 101 in the peripheral direction is preferably at least 30%, at least 50%, at least 70%, at least 80%, at least 90%, or at least 95%. An upper limit of the ratio is not more than 100%.

When the plasma actuator 200 is provided plurally at one spot in the peripheral direction, the plasma actuators 200 is preferably provided at substantially the same position in the longitudinal direction. “Substantially the same position” may refer to the same position where the induced flow 207 merges.

When the cylindrical housing 101 is viewed from the second opening, the plasma actuators 200 are preferably provided in rotational symmetry. For example, the plasma actuators 200 are preferably provided in two-fold symmetry to six-fold symmetry.

Further, the plasma actuator 200 is preferably arranged over the entire circumference of the cylindrical housing 101 in the peripheral direction. By such an arrangement, it is possible to generate the induced flow 207 containing the ozone in the same direction and at the same time. As a result, the induced flow 207 inside of the cylindrical housing 101 merges to generate the airflow 209 with a higher propulsive force.

Since the airflow 209 has a higher propulsive force, the propulsive force of the airflow 213 containing the active oxygen generated by the irradiation of ultraviolet rays also increases. As a result, the treatment amount of gas per unit time increases, thereby enabling an improvement in gas treatment efficiency.

Hereinafter, the plasma actuator 200 will be described in detail.

### First Electrode and Second Electrode

Materials constituting the first electrode and the second electrode are not particularly limited as long as they have excellent conductivity. For example, metals such as copper, aluminum, stainless steel, gold, silver, and platinum and their plated or deposited materials, conductive carbon materials such as carbon black, graphite, and carbon nanotubes and their composite materials mixed with resins, or the like are usable. The material constituting the first electrode and the material constituting the second electrode may be the same or different from each other.

Among these materials, aluminum, stainless steel, or silver, which exhibits minimal corrosion and excellent discharge uniformity, is preferable.

Further, the first electrode and the second electrode may take the form of a flat plate, wire, needle, or the like without any restriction. The first electrode preferably takes the form of a flat plate. Further, the second electrode preferably takes the form of a flat plate. In a case where at least one of the first electrode and the second electrode takes the form of a flat plate, the flat plate preferably has an aspect ratio (the length of the long side/the length of the short side) of at least 2.

The second electrode 201 is preferably an embedded electrode so that plasma is not generated from the second electrode 201. For example, as shown in FIG. 2A, the second electrode is preferably embedded in the dielectric 203 without exposing its edge part. Note that the second electrode 201 here may be embedded only if an induced flow is not generated from its edge part, and its surface may be exposed to the surface of the plasma actuator 200. If the induced flow is generated also from the edge part of the second electrode 201, the direction becomes opposite to that of an induced flow from the first electrode 205, potentially disrupting an airflow in the housing and adversely affecting the longevity of active oxygen.

### Dielectric

A material constituting the dielectric is not particularly limited as long as it has high electric insulating performance. Resins such as polyimide, polyester, fluorocarbon resin, silicone resin, acrylic resin, and phenol resin, glass, ceramics, and their composite materials mixed with resins, or the like are, for example, usable.

Further, in a case where the plasma actuator is arranged over the entire circumference on the inner surface of the cylindrical housing in the peripheral direction as shown in FIGS. 2A to 2F and FIG. 3, a resinous dielectric with bendability, such as polyimide and silicone resin, is preferable. Particularly, a silicone resin is more preferable due to its flexibility and bendability, enabling it to be arranged with high followability even for a housing with a complicated shape.

### Plasma Actuator

The plasma actuator is not particularly limited as long as the first electrode and the second electrode are provided via the dielectric and an induced flow representing a unidirectional jet flow containing ozone is capable of being generated by the application of a voltage between both the electrodes.

In the plasma actuator, plasma is more easily generated as the shortest distance between the first electrode and the second electrode is smaller. Therefore, the thinner the film thickness of the dielectric within the range where electric insulation breaks down does not occur, the more preferable it is. The film thickness may be in the range from 10 μm to 1,000 μm and preferably in the range from 10 μm to 200 μm. Further, the shortest distance between the first electrode and the second electrode is preferably not more than 200 μm. The shortest distance is more preferably in the range from 50 μm to 200 μm.

An aspect of the cross-sectional structure of the plasma actuator 200 is shown in FIG. 2C. The plasma actuator is a so-called dielectric barrier discharge (DBD) plasma actuator (that will be simply called “DBD-PA” below depending on circumstances) in which the exposed electrode (that will also be called the “first electrode” below) 205 with its end surface exposed is provided on one surface (that will also be called the “first surface” below) of the dielectric 203, and in which the second electrode 201 is provided on the surface (that will also be called the “second surface” below) on the side opposite to the first surface. In FIG. 2C, sign 210 shows a dielectric substrate to embed the second electrode 201 in the thickness direction of the plasma actuator to prevent the generation of an induced flow from the end surface of the second electrode. Further, the application of a voltage to both the first electrode and the second electrode is possible with a power supply 211.

In the plasma actuator 200, the first electrode 205 and the second electrode 201, which are arranged via the dielectric 203, are, for example, arranged diagonally opposite to each other. By the application of a voltage between these electrodes (both the electrodes) from the power supply 211, a dielectric barrier discharge oriented from the first electrode 205 toward the second electrode 201 is generated. Then, plasma 208 is generated along an exposed part (a part not coated with the first electrode) 203-1 of the first surface of the dielectric 203 from an edge part 204 of the first electrode 205 toward the extending direction of the second electrode.

At the same time, an air-sucking flow oriented from the internal space of the housing toward to the electrodes is also generated. Electrons in the surface plasma 208 collide with oxygen molecules in the air, and dissociate the oxygen molecules to generate oxygen atoms. After the generated oxygen atoms collide with undissociated oxygen molecules, ozone is generated.

Accordingly, an induced flow 207 containing the concentrated ozone is generated along the surface of the dielectric 203 from the edge part 204 of the first electrode 205 by the operation of a jet-like flow by the surface plasma 208 and the air-sucking flow.

That is, the plasma actuator comprises the first electrode 205, the dielectric 203, and the second electrode 201 laminated together in this order, and the first electrode 205 is an exposed electrode provided on the first surface of the dielectric 203. Then, when a voltage is applied between the first electrode 205 and the second electrode 201, the plasma actuator generates a dielectric barrier discharge oriented from the first electrode 205 toward the second electrode 201, and blows out an induced flow toward the first direction representing one direction along the first surface of the dielectric 203 from the first electrode 205.

More specifically, the plasma actuator generates a dielectric barrier discharge oriented from the edge part 204 on one side of the first electrode 205 toward the second electrode 201, and blows out an induced flow representing a unidirectional jet flow in the first direction along the first surface of the dielectric 203 from the edge part 204 on the one side of the first electrode 205.

Further, the second electrode 201 extends in the blowing-out direction (first direction) of an induced flow in one cross section of the plasma actuator in the thickness direction.

More specifically, for example, the plasma actuator comprises the dielectric 203. When viewed in cross section in the thickness direction, the first electrode 205 and the second electrode 201 are arranged diagonally opposite to each other via the dielectric 203 in the thickness direction of the plasma actuator. The first electrode 205 is provided to coat a part of the first surface of the dielectric 203, and the first surface of the dielectric has the exposed part 203-1 not coated with the first electrode 205. At least a part of the exposed part 203-1 and the second electrode 201 overlap each other.

By the application of a voltage between the first electrode and the second electrode, an induced flow containing ozone is generated along the exposed part of the dielectric overlapping the second electrode 201 in the cross section (FIG. 2C) in the thickness direction, originating from the edge part 204 of the first electrode 205 on the side of the first direction.

The induced flow results in, for example, a wall-surface jet flow along the exposed part 203-1, and easily supplies concentrated ozone in a specific direction. The length of the exposed part 203-1 in the direction of the induced flow (that is, the length from the edge part 204 of the first electrode on the side of the first direction to the edge part of the first surface of the dielectric) is not particularly limited but is preferably in the range from 0.1 mm to 50 mm, more preferably in the range from 0.5 mm to 20 mm, and still more preferably in the range from 1 mm to 10 mm.

The overlap between the first electrode 205 and the second electrode 201 of the plasma actuator serving as an ozone generation device will be described with reference to FIG. 2C. FIG. 2C is a cross-sectional view of the plasma actuator.

When the first electrode 205 and the second electrode 201 arranged diagonally opposite to each other are viewed from the upper side of the cross-sectional view, the edge part 204 of the first electrode may be present in the formed area of the second electrode 201 via the dielectric. That is, the first electrode and the second electrode may be provided to overlap each other via the dielectric. In this case, it is preferable to prevent insulation from breaking down in the area, where the first electrode and the second electrode overlap each other via the dielectric, during the application of a voltage.

FIG. 2C shows an aspect in which the first electrode and the second electrode overlap each other via the dielectric. In the cross section of the plasma actuator in the thickness direction, it is assumed that the edge part 204 of the first electrode on the side of the first direction is an edge part A, and that the edge part of the second electrode on the side of a second direction opposite to the first direction is an edge part B. At this time, the edge part B is preferably positioned on the side of the second direction relative to the edge part A.

The first electrode and the second electrode overlap each other via the dielectric as described above, thus enabling stable occurrence of plasma and an induced flow.

Further, since the first electrode and the second electrode are arranged diagonally opposite to each other via the dielectric 203, the edge part B is positioned on the side of the first direction relative to the edge part of the first electrode on the side opposite to the edge part A. Thus, the occurrence of an induced flow from the edge part of the first electrode on the side opposite to the edge part A is suppressed.

Next, an aspect in which the first electrode and the second electrode do not overlap each other via the dielectric will be described. Assuming that the edge part 204 of the first electrode on the side of the first direction is an edge part A and the edge part of the second electrode on the side of the second direction opposite to the first direction is an edge part B in the cross section of the plasma actuator in the thickness direction, the edge part B is preferably positioned on the side of the first direction relative to the edge part A.

In a case where the first electrode and the second electrode do not overlap each other via the dielectric as described above, it is preferable to relatively increase the voltage applied between both the electrodes in order to compensate for the weakening of an electric field caused when the shortest distance between the electrodes relatively becomes large.

Further, in another preferred aspect, the edge part A and the edge part B align with each other in the thickness direction of the dielectric in the cross section of the plasma actuator in the thickness direction. This aspect refers to, for example, an aspect in which the edge part A and the edge part B are opposed to each other at the shortest distance via the dielectric, and the first electrode and the second electrode either do not overlap each other via the dielectric or do not separate from each other. Thus, the energy applied between both the electrodes is efficiently used to generate an induced flow.

Assuming that an overlapping length is considered positive, the overlap between the edge part of the first electrode and the edge part of the second electrode is preferably in the range from −100 μm to +1,000 μm, more preferably in the range from −0 μm to +200 μm, and still more preferably at 0 μm when viewed from the top of the cross-sectional view.

The thicknesses of both the first electrode and the second electrode are not particularly limited but may be in the range from 10 μm to 1,000 μm. When the thicknesses of the electrodes are at least 10 μm, the resistances of the electrodes become low, making it easier for plasma to be generated. When the thicknesses of the electrodes are not more than 1,000 μm, electric field concentration occurs more easily, making it easier for plasma to be generated.

The widths of both the first electrode and the second electrode are not particularly limited but may be at least 1,000 μm.

In the plasma actuator, an induced flow is not preferably generated from edge parts other than the edge part A of the first electrode defined as described above. Therefore, the edge parts other than the edge part A may be coated with the dielectric. In this manner, a unidirectional jet flow is generated even when the first electrode and the second electrode overlap each other in a Y-axis direction. Further, the shapes of the electrodes may be controlled so that an induced flow is not generated from the edge parts other than the edge part A according to the relationship between the first electrode and the second electrode. For example, in a case where the electrodes have the form of a rectangle, the first electrode and the second electrode may have the same length or the first electrode may be longer than the second electrode in a Z-axis direction (a direction perpendicular to the blowing-out direction of an induced flow from the edge part A). According to such an aspect, the direction of an induced flow is easily controlled.

As shown in FIG. 2D, the first electrode of the plasma actuator may be partially embedded in the dielectric 203 as long as it is exposed to the surface of the dielectric 203.

As shown in FIG. 2E, the second electrode of the plasma actuator may be embedded in the cylindrical housing 101. Further, as shown in FIG. 2F, the second electrode of the plasma actuator may be arranged outside of the cylindrical housing 101.

As shown in FIG. 2C, the induced flow 207 containing ozone flows in the flowing direction of a jet-like flow generated by the surface plasma along the exposed part 203-1 of the first surface of the dielectric 203 from the edge part 204 of the first electrode 205, that is, in the direction along the exposed part 203-1 of the first surface of the dielectric from the edge part 204 of the first electrode 205. This induced flow is a flow of gas containing concentrated ozone with a speed of approximately several meters per second to several tens of meters per second.

The voltage applied between the first electrode 205 and the second electrode 201 of the plasma actuator is not particularly limited as long as it enables the plasma actuator to generate plasma. Further, a DC voltage or an AC voltage may be applied, but the AC voltage is preferably applied. Further, a pulse voltage may be applied as the voltage.

Moreover, the amplitude and frequency of the voltage may be appropriately set in order to adjust the flow velocity of an induced flow and ozone concentration in the induced flow. In this case, the amplitude and frequency of the voltage may be appropriately selected from the viewpoint of generating ozone concentration, which is necessary for generating an effective active oxygen concentration or effective active oxygen amount according to the purpose of treatment, in the induced flow, supplying generated active oxygen to an airflow while maintaining an effective active oxygen concentration or effective active oxygen amount according to the purpose of treatment, or the like.

For example, in a case where the applied voltage is an AC voltage, the difference between the maximum voltage and the minimum voltage of the AC voltage may be in the range from 0.1 kVpp to 100 kVpp. Further, the frequency of the voltage may be preferably at least 1 kHz and more preferably in the range from 10 kHz to 100 kHz.

In a case where the voltage is an AC voltage, the waveform of the AC voltage is not particularly limited, and a sine wave, a rectangular wave, a triangular wave, or the like may be employed. However, the rectangular wave is preferable from the viewpoint of a fast rising voltage.

The duty ratio of the voltage is also appropriately selectable, but the voltage preferably rises fast. Preferably, the voltage is applied so that the rate of voltage rise, which reaches from the bottom to the top of the amplitude of a wavelength, becomes at least 10,000,000 V per second.

Note that a value obtained by dividing the amplitude of the voltage applied between the first electrode 205 and the second electrode 201 by the film thickness of the dielectric 203 (voltage/film thickness) is preferably at least 10 kV/mm.

### Ozone Decomposition Device

The gas treatment device comprises the ozone decomposition device 206. The ozone decomposition device decomposes ozone contained in the airflow 209 to generate active oxygen in the airflow 209. An example of an ozone decomposition device includes one that is capable of acting on ozone contained in an airflow and decomposing the ozone. The ozone decomposition device is preferably one that is capable of decomposing ozone without disrupting an airflow.

The arrangement of the ozone decomposition device is not particularly limited as long as it is capable of achieving the effect of the present invention. However, the ozone decomposition device is preferably provided at the center of the cylindrical housing so as not to hinder the travel of an induced flow.

Further, even in a case where the ozone decomposition device is attached to the inner wall of the cylindrical housing, the arrangement of the ozone decomposition device is not limited as long as it is capable of achieving the effect of the present disclosure. However, the ozone decomposition device is preferably embedded in the housing so as not to become an obstacle to an induced flow, and structured so as not to protrude from the inner surface of the housing.

The ozone decomposition device is preferably at least one device selected from a group consisting of an ultraviolet light source irradiating an airflow with ultraviolet rays to generate active oxygen in the airflow, a heating device heating an airflow to generate active oxygen in the airflow, and a humidifying device humidifying an airflow to generate active oxygen in the airflow. The ozone decomposition device may be a combination of these devices. For example, the ozone decomposition device may be a device heating an airflow while irradiating the airflow with ultraviolet rays or a device humidifying the inside of the housing while irradiating an airflow with ultraviolet rays and heating the same. The ozone decomposition device is more preferably an ultraviolet light source. That is, the gas treatment device according to an aspect of the present disclosure preferably comprises, as the ozone decomposition device, at least an ultraviolet light source irradiating an airflow with ultraviolet rays to generate active oxygen in the airflow.

The respective devices will be described below.

### Ultraviolet Light Source and Ultraviolet Rays

The ultraviolet light source is not particularly limited as long as it is capable of exciting ozone and irradiating ultraviolet rays enabling the generation of active oxygen. Further, the ultraviolet light source is not particularly limited as long as it has the wavelength and illumination of ultraviolet rays necessary for obtaining effective active oxygen concentration and an effective active oxygen amount according to the purpose of treatment.

For example, the peak value of the light absorption spectrum of ozone is 260 nm. From this point of view, the peak wavelength of the ultraviolet rays is preferably in the range from 220 nm to 310 nm, more preferably in the range from 253 nm to 285 nm, and still more preferably in the range from 253 nm to 266 nm.

As a specific ultraviolet light source, a low-pressure mercury lamp in which mercury is enclosed in quartz glass together with inactive gas such as argon and neon, a cold-cathode tube ultraviolet lamp (UV-CCL), an ultraviolet LED, or the like is usable. The wavelength of the low-pressure mercury lamp or the cold-cathode tube ultraviolet lamp may be selected from options such as 254 nm. On the other hand, the wavelength of the ultraviolet LED may be selected from options such as 265 nm, 275 nm, and 280 nm from the viewpoint of output performance.

### Heating Device

The heating device is not particularly limited as long as it is capable of exciting ozone in the airflow 209 and providing heat energy enabling the generation of active oxygen. The thermal decomposition of ozone starts at approximately 100° C. From this point of view, a device capable of heating the airflow 209 to approximately 120° C. is preferable. On the other hand, if the heating temperature of the heating device exceeds 120° C., there may be a case where thermal degradation such as melting and decomposition occurs in the housing depending on the material of the housing. Therefore, the heating temperature is preferably not more than 200° C. The heating temperature is preferably in the range from 100° C. to 140° C. and more preferably in the range from 110° C. to 130° C.

The heating device is not particularly limited but may be, for example a device including a heat source (heat supply unit) that supplies heat or a device not including a heat source (heat supply unit). Specifically, a ceramic heater, a cartridge heater, a sheathed heater, an electric heater, an oil heater, or the like is usable as a heating device including a heat supply unit. In the case of a device including a metal heating element, the heating element is preferably made of a material such as a nichrome alloy and tungsten having excellent oxidation resistance. As a heating device not including a heat supply unit, a device that heats the airflow 209 through dielectric heating (such as microwave heating, electronic heating, high-frequency heating, and radio frequency heating) is usable. A cartridge heater is preferably used.

### Humidifying Device

The humidifying device is not particularly limited as long as it is capable of humidifying the inside of the housing to cause water to be contained in the airflow 209 and decomposing ozone in the airflow with the water to generate active oxygen in the airflow. Here, the humidifying process involves giving moisture to a target, and the form of the moisture is not particularly limited but may be at least one selected from a group including gas, a liquid, and a solid.

Further, known water is arbitrarily usable as water for giving moisture, and this water may contain substances other than water.

The humidifying device is not particularly limited but is, for example, a vaporizing type humidifying device or a mist type humidifying device.

In order to suppress humidity near the plasma actuator, the humidifying device is preferably one that has directivity with respect to a moisture supply direction (that will also be simply called directivity below). With the directivity of the humidifying device, it is possible to efficiently humidify the vicinity of the airflow 209 or the vicinity of the surface of an object to be treated without increasing humidity near the plasma actuator.

In order to make the humidifying device have the directivity, a known method is suitably available. For example, a method in which an airflow is generated by a fan provided so as not to disrupt an induced flow and the airflow 209 and moisture is supplied to the direction of the airflow, a method in which an appropriate pressure is given to moisture by an air pump or the like to inject the moisture in a desired direction, or the like, is available. In order to prevent the induced flow and the airflow 209 from being disrupted, the humidifying device is preferably directed in the same direction (first direction) as the induced flow and the airflow 209.

### Arrangement of Plasma Actuator and Ozone Decomposition Device

In the gas treatment device 100, the position of the plasma actuator that generates an induced flow containing ozone is not particularly limited as long as the plasma actuator and the ozone decomposition device are arranged so that active oxygen in the airflow 209, which is generated by ultraviolet rays irradiated from the ultraviolet light source 206 representing the ozone decomposition device, maintains an effective active oxygen concentration or effective active oxygen amount according to the purpose of treatment. The same applies to a case where the ozone decomposition device is a heating device or a humidifying device.

Further, for example, the plasma actuator and the ozone decomposition device are preferably arranged so that an airflow containing active oxygen flows to the outside of the gas treatment device from the second opening. By such an arrangement, it is possible to treat gas as an object to be treated, which is present in the outflow direction of an airflow containing active oxygen, by the airflow containing the active oxygen flowing to the outside of the gas treatment device from the second opening.

Further, the distance between the ozone decomposition device and the plasma actuator differs depending on the purpose of treatment, and therefore is not necessarily stipulated. For example, the distance between the surface of the dielectric of the plasma actuator opposed to the ozone decomposition device and the ozone decomposition device is preferably not more than 15 mm, more preferably not more than 10 mm, and still more preferably not more than 4 mm. However, it is not necessary to arrange the plasma actuator at a place within approximately 15 mm from the ozone decomposition device. The distance between the ozone decomposition device and the plasma actuator is not particularly limited as long as active oxygen in an airflow has an effective concentration according to the purpose of treatment in relation to elements enabling the decomposition of ozone such as the illumination and wavelength of ultraviolet rays.

Further, in the plasma actuator, an ozone generation amount per unit time in a state in which ozone in the airflow 209 is not decomposed by the ozone decomposition device is, for example, preferably at least 15 μg/min. The ozone generation amount is more preferably at least 30 μg/min. An upper limit of the ozone generation amount is not particularly limited but is, for example, not more than 1,000 μg/min.

The flow velocity of an induced flow or the airflow 209 may be, for example, a rate at which the airflow 209 is treated by the gas treatment device 100 while advancing inside of the housing, and may be a rate at which the airflow 209 is capable of treating gas as an object to be treated at an airflow advancing destination representing the outside of the housing. The flow velocity is, for example, in the range from approximately 0.01 m/s to 100 m/s.

The concentration of ozone in an induced flow generated from the plasma actuator as described above or the flow velocity of the induced flow may be controlled by the thicknesses or materials of the electrodes or the dielectric, the type, amplitude, frequency of an applied voltage, or the like.

### Housing and Opening Parts

The gas treatment device according to the present disclosure comprises: the cylindrical housing 101 having the first opening and the second opening on the side opposite to the first opening; the plasma actuator 200 arranged inside of the housing; and the ozone decomposition device 206.

Preferred embodiments of this aspect will be described below, but the arrangement of the first electrode and the second electrode in the housing is appropriately selectable so that, for example, active oxygen generated in the airflow 209 maintains an effective active oxygen concentration or effective active oxygen amount according to the purpose of treatment.

The cylindrical housing 101 may be designed so that the plasma actuator is attached inside of the housing 101, and that air does not flow in from any area other than the first opening. Therefore, the shape of a cross section, an inner diameter, an outer diameter, the ratio of the inner diameter to the outer diameter, the inner diameter, the outer diameter, and the ratio of the inner diameter to the outer diameter from the first opening to the second opening, the change amount of a cross-sectional shape, the coaxiality between the first opening and the second opening, the bending angle of the cylindrical housing 101, the material of the housing, or the like is not particularly limited.

The cylindrical housing 101 is preferably configured so as not to disrupt a laminar flow of an induced flow containing ozone and the airflow 209 or an induced flow containing active oxygen and the airflow 209. For example, the cylindrical housing 101 preferably has a structure in which there is no obstacle between a point on the extension of the direction along the exposed part 203-1 of the first surface of the dielectric from the edge part of the first electrode of the plasma actuator and the second opening.

The length of the cylindrical housing 101 in the longitudinal direction is appropriately selectable. However, an airflow flowing in from the first opening is easily straightened by an induced flow generated by the plasma actuator. Therefore, the distance between the first opening and the plasma actuator is preferably set to be longer than the distance between the second opening and the plasma actuator.

The cross-sectional shape of the cylindrical housing 101 of the gas treatment device in the direction perpendicular to a direction from the first opening toward the second opening is appropriately selectable, including polygonal shapes such as rectangles, circles, ellipses, combinations of circles and polygonal shapes, or the like. For example, the cross-sectional shape is preferably a circular shape or a square shape. That is, the cylindrical housing preferably has a cylindrical shape or a rectangular cylindrical shape. Since a laminar flow of an induced flow containing ozone could be disrupted, the cylindrical housing preferably has a shape where the cross-sectional shape or the phase of the cross-sectional shape does not change during the advancement of the airflow 209 from the first opening toward the second opening.

In order to reduce an opportunity for the airflow 209 to come into contact with the inner wall of the housing, the inner diameter of the cylindrical housing 101 preferably remains constant from the first opening toward the second opening 103.

The cylindrical housing 101 may be configured with a material and a thickness, such as metal, ceramics, and resin that does not deform due to its own weight, in order to prevent disruption in the advancement of an induced flow and the airflow 209 from the first opening to the second opening. The cylindrical housing 101 is preferably configured with a highly-insulative material and a thickness in order to prevent leakage from the electrodes of the plasma actuator to the outside.

A manufacturing method for the cylindrical housing 101 is preferably a method for preventing the occurrence of places where air from the outside flows other than the first opening. Specifically, the cylindrical housing 101 may be a hollow housing molded by injection molding or extrusion molding, a housing obtained by hollowing out a solid housing, which was manufactured in the same manner, through a process such as cutting, a housing obtained by rolling up a sheet and seamlessly bonding joining parts together, or the like.

The length of the cylindrical housing from the first opening to the second opening may be appropriately changed according to the purpose of treatment and is not particularly limited. However, the length is preferably in the range from 3 mm to 1,000 mm, more preferably in the range from 5 mm to 100 mm, and still more preferably in the range from 10 mm to 50 mm.

For the first opening, the size of its opening part, the relative position of the opening with respect to the center of the cylindrical housing, and the shape of the opening are not limited as long as air is allowed to flow in from the outside of the first opening when gas inside of the housing transforms into an airflow and moves toward the second opening due to an induced flow generated by the plasma actuator 200. Further, a lid that controls the shape and size of the first opening may be provided as long as the effect of this aspect is not hindered. Particularly, in order to suppress the turbulence of air, the first opening preferably has the same shape as the cross-sectional shape of the inner periphery of the cylindrical housing.

The inner diameter of the first opening may be appropriately changed according to the purpose of treatment and is not particularly limited. In order to stabilize an induced flow generated by the plasma actuator 200, it is preferable to increase a flow rate, which contributes to the flow of air sucked into the plasma actuator 200, among the flow rates of air flowing in from the first opening. Accordingly, a maximum diameter of the opening part of the first opening is preferably in the range from 5 mm to 100 mm and more preferably in the range from 10 mm to 50 mm.

For the second opening, the size of its opening part, the relative position of the opening with respect to the center of the cylindrical housing, the shape of the opening part, and the relative position of the opening part with respect to an object to be treated are not limited as long as the airflow 209 generated from the plasma actuator 200 flows to the outside of the second opening of the cylindrical housing 101. Further, a lid that controls the shape and size of the second opening may be provided as long as the effect of this aspect is not hindered. Particularly, in order to suppress the turbulence of air, the second opening preferably has the same shape as the cross-sectional shape of the inner periphery of the cylindrical housing. Further, the inner diameter of the second opening may be appropriately changed according to the purpose of treatment and is not particularly limited. However, a maximum inner diameter of the second opening may be preferably in the range from 5 mm to 100 mm and more preferably in the range from 10 mm to 50 mm.

For example, in the second opening, the illumination of ultraviolet rays is not particularly limited but is preferably set to decompose ozone contained in an induced flow, generate active oxygen in an airflow, and generate an effective active oxygen concentration or effective active oxygen amount according to the purpose of treatment. Specifically, the illumination of ultraviolet rays in the second opening is, for example, preferably at least 40 μW/cm², more preferably at least 100 μW/cm², still more preferably at least 400 μW/cm², and particularly preferably at least 1,000 μW/cm². An upper limit of the illumination is not particularly limited but may be, for example, not more than 10,000 μW/cm².

The plasma actuator 200 may be configured to be continuous or cut off at a plurality of places in the peripheral direction or the longitudinal direction of the cylindrical housing as long as it is capable of generating the airflow 209 containing ozone toward the second opening. For example, from the viewpoint of generating a straightened airflow, the plasma actuator is preferably uniformly provided in the cross section of the housing perpendicular to the longitudinal direction as an aspect.

Particularly, as a structure that prevents the airflow from coming into contact with the inner wall inside of the cylindrical housing 101 as much as possible and makes it possible to increase the propulsive force of the airflow 209, as shown in FIG. 3, the plasma actuator is preferably configured to be continuous on the inner peripheral surface over the entire circumference in the peripheral direction. Further, in order to enhance the effect of gas treatment, the plasma actuator is preferably arranged at a plurality of places (for example, two to four places) in the direction (longitudinal direction) from the first opening toward the second opening between the first opening and the second opening of the cylindrical housing, from the viewpoint of increasing the propulsive force or increasing active oxygen concentration inside of the cylindrical housing.

Further, it is also possible to bundle a plurality of the cylindrical gas treatment devices according to the present invention together in use.

Note that the “effective active oxygen concentration or effective active oxygen amount” in the present disclosure refers to an active oxygen concentration or active oxygen amount for achieving the treatment of gas inside of the housing or active oxygen concentration or an active oxygen amount for achieving the treatment of gas outside of the housing, and may be appropriately adjusted according to purposes using the thicknesses and materials of the electrodes and the dielectric constituting the plasma actuator, the type, amplitude, and frequency of an applied voltage, the illumination and irradiation time of ultraviolet rays, or the like.

The gas treatment device according to the present disclosure is applicable to various applications implemented by supplying active oxygen to gas as an object to be treated. For example, the gas treatment device according to the present disclosure is applicable also to purposes such as gas sterilization and deodorization.

### Second Aspect

FIGS. 4A to 4C show a gas treatment device according to a second aspect of the present disclosure.

In the gas treatment device according to this aspect, a cylindrical housing 101 itself comprises a dielectric.

In the cross section of the cylindrical housing along an axial direction, a first electrode 205 representing an exposed electrode provided to coat a part of an inner surface is arranged on the inner surface of the housing. Further, a second electrode 201 electrically insulated from the first electrode 205 via the dielectric is arranged outside of the inner surface of the cylindrical housing 101. That is, the gas treatment device according to this aspect differs from the gas treatment device according to the first aspect in that the cylindrical housing is used as a dielectric portion of a plasma actuator.

The entire cylindrical housing does not necessarily need to be made of the dielectric, but it is sufficient for only the portion where the first electrode 205 and the second electrode 201 are electrically insulated from each other, and where an induced flow representing a unidirectional jet flow is generated from the first electrode 205, to be made of the dielectric. That is, portions not affecting the occurrence of an induced flow may be made of materials other than the dielectric. The cylindrical housing 101 is preferably made of the dielectric.

As an example of the arrangement of the first electrode 205, the first electrode 205 is arranged over the entire circumference of the inner peripheral surface of the cylindrical housing 101 in the peripheral direction, and the second electrode 201 is arranged over the entire circumference of the outer peripheral surface of the cylindrical housing 101 in the peripheral direction as shown in FIG. 4B viewed from the side of the second opening of the gas treatment device 100. However, the arrangement of the first electrode 205 is not limited to this, and the first electrode 205 may be arranged at one place or a plurality of places in the peripheral direction.

For the arrangement of the second electrode 201, the second electrode is also preferably provided over the entire circumference in the peripheral direction from the viewpoint of the occurrence efficiency of an induced flow in a case where the first electrode 205 is provided over the entire circumference in the peripheral direction as described above. However, the arrangement of the second electrode 201 is not limited to this, and the second electrode 201 may be arranged at one place or a plurality of places in the peripheral direction as long as an induced flow is generated from at least a part of the first electrode. Moreover, in a case where the first electrode 205 is arranged at one place or a plurality of places, the second electrode 201 may also be arranged at one place or a plurality of places corresponding to the arrangement position(s) of the first electrode.

In this aspect, the cylindrical housing 101 is configured as the dielectric of the plasma actuator. Therefore, the material of the cylindrical housing 101 is a material having high electric insulating performance. As the dielectric, resins such as polyimide, polyester, fluorocarbon resin, silicone resin, acrylic resin, and phenol resin, glass, ceramics, and their composite materials mixed with resins or the like are, for example, usable. Among these materials, a resinous material that hardly catches fire even in a case where a current leaks and that has flexibility is preferable.

The dielectric is more preferably a silicone resin. The silicone resin is capable of achieving both insulating performance and flexibility at a high level.

The first electrode according to this aspect may be formed on the surface of the inner peripheral surface or partially embedded in the cylindrical housing 101 when formed on the inner peripheral surface of the cylindrical housing 101, as long as it is capable of generating an induced flow containing ozone and an airflow 209.

As shown in FIGS. 4A and 4C, the second electrode 201 is formed outside of the inner surface of the cylindrical housing 101, but its position is not particularly limited as long as it is capable of generating an induced flow containing ozone and the airflow 209. Specifically, the second electrode 201 may be, for example, formed on the surface of the outer peripheral surface of the cylindrical housing as shown in FIG. 4A, or may be partially or entirely embedded in the outer peripheral surface of the cylindrical housing as shown in FIG. 4C. Further, in a case where the second electrode is formed on the surface of the outer peripheral surface, the second electrode on the outer peripheral surface may be coated with a substrate such as a dielectric to prevent the generation of an induced flow from the edge part of the second electrode.

Moreover, in a case where the first electrode and the second electrode are formed on the cylindrical housing 101, an operation (cutting or grinding) to change the thickness of the cylindrical housing 101 may be performed at the positions where the electrodes are formed, as long as an induced flow containing ozone is suitably generated.

In this aspect, the shapes, arrangements, or the like of the cylindrical housing, the first opening, the second opening, the first electrode and second electrode of the plasma actuator, an ozone decomposition device such as an ultraviolet light source, and other constituting elements can be configured in the same manner as the contents described in the first aspect, and the dielectric in the first aspect can be rephrased as the cylindrical housing in this aspect. For example, generated active oxygen is appropriately selectable to be actively supplied to the surface region of an object to be treated, while maintaining an effective active oxygen concentration and an effective active oxygen amount according to the purpose of treatment.

Further, the present disclosure provides a gas treatment method using active oxygen, the method comprising:
a step of preparing a gas treatment device, wherein
the gas treatment device comprises
   a cylindrical housing having a first opening and a second opening on the side opposite to the first opening,
   a plasma actuator arranged inside of the housing, and
   an ozone decomposition device,
the plasma actuator comprises a first electrode, a dielectric, and a second electrode,
the dielectric is interposed between the first electrode and the second electrode,
the first electrode and the second electrode are electrically insulated from each other,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the plasma actuator is arranged so that the first direction representing a blowing-out direction of the induced flow is oriented toward the second opening, and causes an airflow containing gas flowing in from the first opening and oriented from the first opening toward the second opening to be generated inside of the housing by the induced flow,
the ozone decomposition device decomposes the ozone contained in the airflow to generate active oxygen in the airflow, and the induced flow results in an airflow containing the active oxygen, whereby
the gas in the airflow containing the active oxygen is treated by the active oxygen, or the airflow containing the active oxygen treats gas as an object to be treated, which is present in an outflow direction of the airflow containing the active oxygen, using the active oxygen.

### EXAMPLES

The present disclosure will be described in further detail using Examples and Comparative Examples below, but the aspects of the present disclosure are not limited to the Examples and Comparative Examples.

### Example 1

### 1. Manufacturing of Gas Treatment Device

An aluminum foil having a longitudinal length of 2.5 mm, a horizontal length of 62.8 mm, and a thickness of 100 μm was pasted onto a first surface of a polyimide sheet (having a longitudinal length of 5 mm, a horizontal length of 62.8 mm, and a thickness of 100 μm) serving as a dielectric, using an adhesive tape to form a first electrode. Further, an aluminum foil having a longitudinal length of 3 mm, a horizontal length of 62.8 mm, and a thickness of 100 μm was pasted onto a second surface of the polyimide sheet on the side opposite to the first surface using an adhesive tape so as to be diagonally opposite to the aluminum foil pasted ono to the first surface to form a second electrode. Moreover, in order to prevent the occurrence of an induced from the second electrode, the second surface including the second electrode was coated with a polyimide tape. In this manner, a plasma actuator was manufactured with the first electrode and the second electrode overlapping each other over a width of 500 μm via the dielectric (polyimide sheet).

Next, an ABS resin sheet (having a longitudinal length of 30 mm, a horizontal length of 62.8 mm, and a thickness of 1 mm) was prepared as a material for a housing 101 of a gas treatment device 100. Then, the plasma actuator previously manufactured was affixed to one surface of the ABS resin sheet. Specifically, the side of the polyimide tape coating the second electrode 201 of the plasma actuator 200 was bonded and fixed to the surface of the ABS resin sheet. Next, the ABS resin sheet was rolled up in a cylindrical shape with the surface where the plasma actuator was affixed positioned on the inner side, thereby manufacturing a cylindrical housing with the plasma actuator fixed over its entire circumference as shown in FIGS. 2A and 2B. The cylindrical housing 101 had a first opening (not shown) and a second opening 103. Note that the length from the first opening to the second opening 103 was set at 30 mm. Further, the length from the first opening to the edge part of the plasma actuator on the side of the first opening was set at 15.0 mm. Moreover, the plasma actuator was fixed so that the direction of a jetted induced flow 207 containing ozone generated from the vicinity of the first electrode 205 was oriented toward the second opening 103.

Subsequently, an ultraviolet lamp 206 (an ultraviolet lamp 206: a cold-cathode tube ultraviolet lamp, product name: UW/9F89/9, manufactured by Stanley Electric Co., Ltd, cylindrical shape having a length of 150 mm and a diameter of 9 mm, peak wavelength of 254 nm) was fixed in the central portion of the housing using a support member not shown inside of the housing 101.

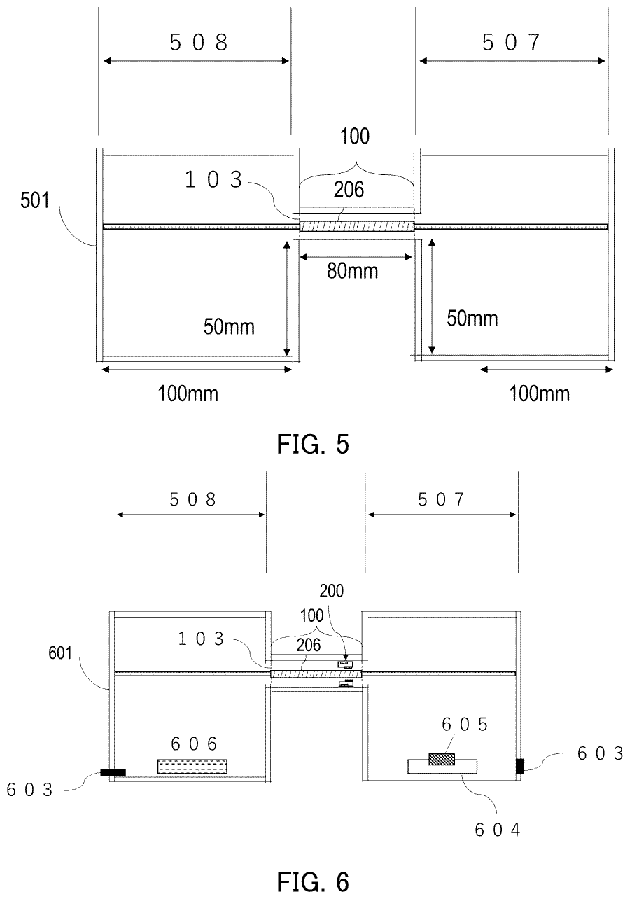

The gas treatment device 100 according to this Example manufactured above was installed in an evaluation device 501 as shown in FIG. 5. In FIG. 5, the frame of the evaluation device 501 was made of acrylic resin. Further, the gas treatment device 100 and the evaluation device 501 are connectable without a gap therebetween, and configured so that air does not enter from the outside.

At the position of the second opening 103 serving as a supply port for active oxygen in the gas treatment device 100, a spectroradiometer (product name: USR-45D, manufactured by Ushio Inc.) was placed to measure the illumination of ultraviolet rays. The measured illumination was 1370 μW/cm² based on the integrated value of the spectrum. At this time, the plasma actuator was not powered on to prevent an affect by the shielding of ultraviolet rays due to ozone generated from the plasma actuator.

Subsequently, the gas treatment device 100 was placed into a sealed container (not shown) having a volume of 1 L to calculate the amount of ozone generated from the plasma actuator 200. The sealed container was equipped with an opening sealable with a rubber plug, thus enabling the suction of internal gas from the opening using an injector. Then, the ultraviolet lamp was not turned on, and a voltage of 2.4 kVpp having a frequency of 80 kHz and a sine waveform was applied to the plasma actuator 200. After 1 minute had elapsed, 100 mL of the gas inside of the sealed container was collected. The collected gas was sucked by an ozone detection tube (product name: 182SB, manufactured by KOMYO RIKAGAKU KOGYO K.K.) to measure the concentration (PPM) of measured ozone contained in an induced flow from the plasma actuator 200. Using the value of the measured ozone concentration, an ozone generation amount per unit time was calculated by the following equation.

$$Amount of ozone generated per unit time ( \frac{mg}{min} ) = Measured ozone concentration ( PPM ) * \frac{Molecular weight of ozone 48}{22.4} * \frac{\frac{273}{273 + Room temperature ( ° C . )}}{10000} * \frac{Gas inside of sealed container ( L )}{Collected gas ( L )} = Measured ozone concentration ( PPM ) * 48 / 22.4 * 273 / ( 273 + 25 ) / 10000 * 0.1 / 1$$

As a result, the ozone generation amount per unit time was 130 μg/min. At this time, an ultraviolet light source was not powered on to prevent an affect by ozone decomposition due to ultraviolet rays irradiated from the ultraviolet light source.

Finally, the ozone generation amount during the operation of both the plasma actuator 200 and the ultraviolet lamp 206 was measured. The operating condition of the plasma actuator 200 was such that 130 μg/min of ozone was generated when only the plasma actuator 200 was operated. Further, the operating condition of the ultraviolet lamp 206 was such that an illumination of 1370 μW/cm² was obtained when only the ultraviolet lamp 206 was operated. As a result, the ozone generation amount was 10 μg/min in a case where both the plasma actuator 200 and the ultraviolet lamp 206 were operated. It appears that 120 μg/min, which corresponds to the reduction amount from 130 μg/min, represents the amount of ozone transformed into active oxygen.

### 2. Manufacturing of Sterilization Rate Measurement Mechanism

In this Example, a test device 601 as shown in FIG. 6 was configured using the evaluation device 501, and sterilization performance by the gas treatment device 100 was evaluated.

The test device 601 is composed of the gas treatment device 100, a fungus-containing-gas preparation unit 507, and a fungus collection unit 508, and the fungus-containing-gas preparation unit 507, the gas treatment device 100, and the fungus collection unit 508 were sequentially installed in this order. Acrylic resin was used in the frames of the fungus-containing-gas preparation unit 507 and the fungus collection unit 508.

Bores 603 having a diameter of 5 mm were provided on wall surfaces in four directions adjacent to the bottom surfaces of the fungus-containing-gas preparation unit 507 and the fungus collection unit 508, ensuring that a gas flow accompanied by the generation of an induced flow from the gas treatment device 100 was not hindered. The bores 603 provided in the fungus-containing-gas preparation unit 507 serve as suction ports, and the bores 603 provided in the fungus collection unit 508 serve as exhaust ports.

### 2-1. Confirmation Test for Active Oxygen

In order to confirm whether active oxygen was generated inside of the housing as a result of the operation of the gas treatment device manufactured according to the above section 1, the following test was conducted. That is, the presence or absence of active oxygen in an airflow flowing out from the second opening 103 was confirmed by making use of the decolorization reaction of a methylene blue solution (“Magnetic Field Effect on the Photocatalytic Reaction with TiO₂ Semiconductor Film,” Journal of the Society of Photography and Technology of Japan, 2006, 69, 4, 271-275).

Specifically, methylene blue (KANTO CHEMICAL CO., INC., highest quality) was mixed with distilled water to prepare a 0.01% methylene blue solution. 15 mL of the methylene blue solution was placed into a petri dish (AB4000 manufactured by EIKEN CHEMICAL CO., LTD., a column shape having a diameter of 88 mm). As a result, a petri dish A containing the methylene blue solution was prepared. The gas treatment device 100 and the petri dish A were arranged so that the surface center of the petri dish A and the center of the second opening of the gas treatment device 100 were opposed to each other at a distance of 1 mm. Note that in this test, only the cylindrical housing 101 and the ultraviolet light source 206 were extracted from the sterilization rate evaluation device shown in FIG. 5 for the confirmation of active oxygen and arranged as described above with respect to the petri dish A to conduct the test.

Next, the ultraviolet lamp was lit up, while applying an AC voltage with a sine waveform having an amplitude of 2.4 kVpp and a frequency of 80 kHz between both electrodes of the plasma actuator, thereby supplying an induced flow flowing out from the opening toward the surface of the liquid for 60 minutes. Note that the ultraviolet lamp was adjusted so that illumination measured at the exposed surface of the dielectric of the plasma actuator on the side opposite to the ultraviolet lamp was 1370 μW/cm² without powering on the plasma actuator.

The methylene blue solution after the supply of the induced flow was transferred from the petri dish to a cell, and a change in the light absorption amount of methylene blue was measured using a spectrophotometer (V-570 manufactured by JASCO Corporation). Since methylene blue exhibits strong absorption at a wavelength of 664 nm, the degree of the decolorization of methylene blue is calculatable from a change in absorbance at the wavelength. In this test, when only distilled water was first placed into the reference cell and a 0.01% methylene blue solution before the supply of the induced flow was placed into the sample cell and measured, the absorbance was 2.32 Abs. On the other hand, the absorbance of the methylene blue solution after the supply of the induced flow was 0.27 Abs. Therefore, the reduction rate of the absorbance was 88% (((2.32−0.27)/2.32)×100).

### 2-2. Treatment (Sterilization) Test

Using the test device 601 in which the gas treatment device 100 was installed, a sterilization test for E. coli was conducted in the following procedure. Note that all the equipment used in this sterilization test were those having undergone high-pressure steam sterilization using an autoclave. Further, this sterilization test was conducted inside of a clean bench.

First, E. coli (product name “KWIK-STIK (E. coli (Escherichia coli) ATCC8739)”, produced by Microbiologics, Inc.) was placed into an Erlenmeyer flask containing an LB medium (prepared by dissolving 200 mL of distilled water in a mixture of 2 g of tryptone (product name “Bacto Tryptone,” produced by Life Technologies Japan Ltd.), 1 g of yeast extract (product name “Yeast Extract,” produced by Life Technologies Japan Ltd.), and 1 g of sodium chloride (product name “sodium chloride, highest quality,” produced by Kishida Chemical Co., Ltd.)). Subsequently, the Erlenmeyer flask was shaken and incubated using an incubator shaker (TA-25R-3F, manufactured by Takasaki Kagaku Kikai Co., Ltd.,) for 48 hours at 37° C. and at 80 rpm to obtain an E. coli solution. A viable cell count of the obtained E. coli solution was 9.2×10⁹ (CFU/mL).

20 mL of the produced E. coli solution was placed into a petri dish, and the petri dish was arranged at the bottom of the fungus-containing-gas preparation unit 507 to be set as an E. coli solution petri dish 604. As a method for generating gas containing E. coli, a method for forming mist of the coli solution in the fungus-containing-gas preparation unit 507 was employed. That is, a method for forming mist of the coli solution using an ultrasonic sprayer (manufactured by REN HE Company) was employed. The ultrasonic sprayer consists of a substrate (not shown) to perform control and a vibrator 605 connected to the substrate by a wire. The vibrator is composed of ABS resin, silicone resin, ceramics material, and the ceramics material has a circular sheet shape having a diameter of 16 mm and is equipped with numerous minute holes measuring 5 μm in size from its upper surface to lower surface. Since the ceramic material has piezoelectric properties, ultrasonic vibration is generated by the application of a voltage. When ultrasonic waves are generated with the lower surface of the vibrator coming into contact with the liquid surface of the coli solution, the coli solution forms fine liquid droplets as it passes through the minute holes so as to be absorbed, and mist is ejected from the upper surface of the vibrator.

In the fungus collection unit, a stamp medium 606 (Petan Check 25 PT1025, produced by EIKEN CHEMICAL CO., LTD.) was provided at its bottom.

Subsequently, the ultrasonic sprayer and the gas treatment device were operated at the same time, and this time point was set as 0 seconds. A 5 V DC voltage was applied to the ultrasonic sprayer. By making use of the generation of an induced flow inside of the gas treatment device 100 through the irradiation of ultraviolet rays resulting from the application of a 7 V DC voltage to the gas treatment device 100 and the ultraviolet light source 206, air containing E. coli entering the gas treatment device 100 from the fungus-containing-gas preparation unit 507 was treated. This test was conducted for 20 seconds and completed when the gas treatment device and the ultrasonic sprayer were stopped at the same time.

After the test was completed, the stamp medium of the fungus collection unit 508 was placed into a constant temperature bath (product name: IS600, manufactured by Yamato Scientific Co., Ltd.) and incubated for 24 hours at 37° C. to obtain a sample No. 1. The number of generated colonies was counted to obtain a viable cell count after the sterilization treatment. As a result, the viable cell count associated with the sample No. 1 was 4 (CFU).

Next, except that the treatment by the gas treatment device was not conducted, an incubation test was conducted as in the case of the sample No. 1 to obtain a sample No. C1, and the number of colonies was counted to calculate a viable cell count. As a result, the viable cell count associated with the sample No. C1 was 196 (CFU).

Accordingly, the sterilization rate of E. coli by the gas treatment device according to this test was 98.0% (=(196−4)/196).

### Example 2

A gas treatment device was manufactured in the same manner as Example 1 except that the material of the dielectric of a plasma actuator was a silicone resin. The gas treatment device was then evaluated. The evaluation results are shown in Table 1.

### Example 3

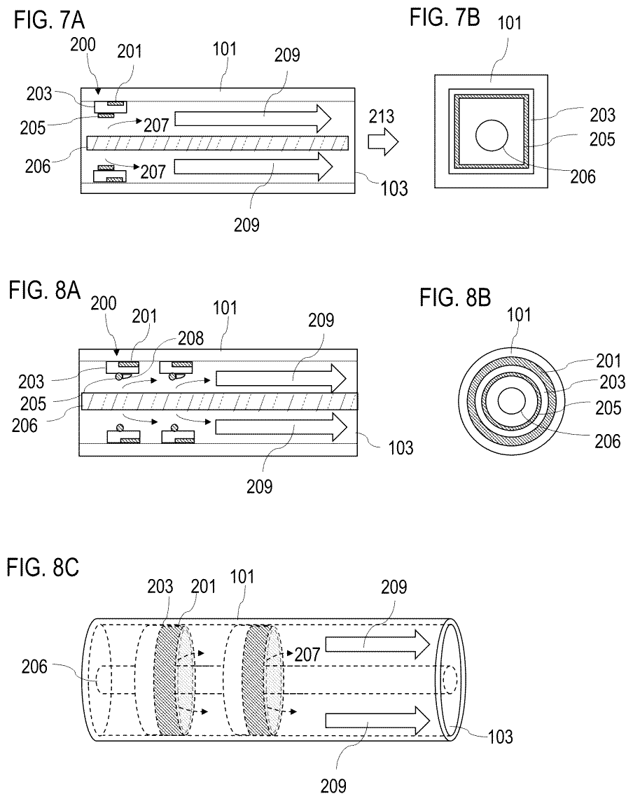

A gas treatment device was manufactured in the same manner as Example 2 except that the cross-sectional shape of a cylindrical housing 101 was changed to a quadrangular (square) cross-sectional shape as shown in FIGS. 7A (a cross-sectional view in a longitudinal direction) and 7B (a view when viewed from a second opening). The gas treatment device was then evaluated. The evaluation results are shown in Table 1. Note that diameters in Table 1 represent the lengths of the diagonal lines of cross-sectional shapes.

### Example 4

A gas treatment device was manufactured in the same manner as Example 2 except that plasma actuators were formed in two rows on the inner peripheral surface of a cylindrical housing 101 as shown in FIGS. 8A (a cross-sectional view in a longitudinal direction), 8B (a view when viewed from a second opening), and 8C (a perspective view). The gas treatment device was then evaluated. The evaluation results are shown in Table 1.

### Example 5

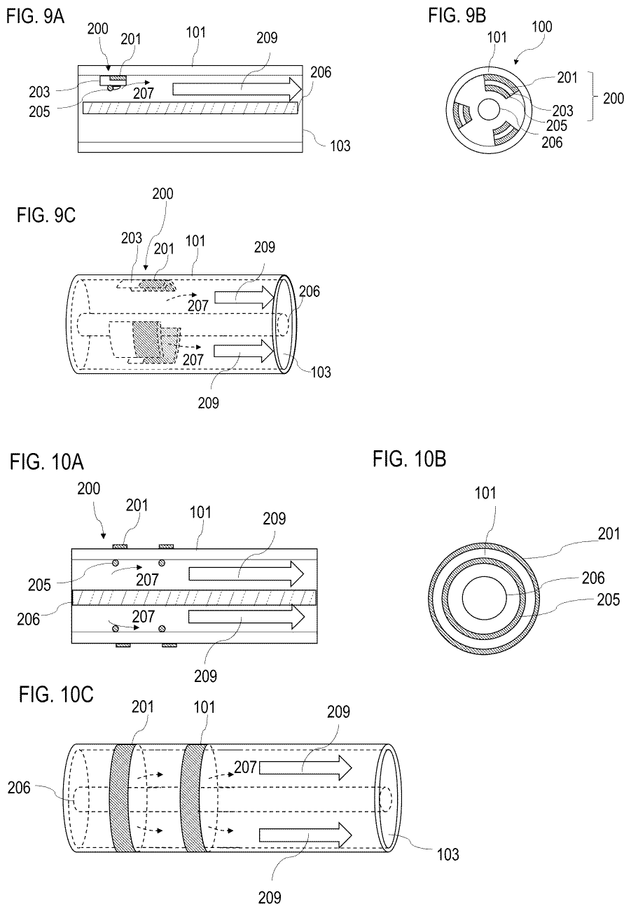

A gas treatment device was manufactured in the same manner as Example 2 except that a plasma actuator was not installed over an entire circumference and was arranged to be discontinuous on the inner peripheral surface of a cylindrical housing 101 as shown in FIGS. 9A (a cross-sectional view in a longitudinal direction), 9B (a view when viewed from a second opening), and 9C (a perspective view). The gas treatment device was then evaluated. The evaluation results are shown in Table 1.

### Example 6

In this Example, a gas treatment device was manufactured in the same manner as Example 2 except that a cylindrical housing 101 was used as the dielectric of a plasma actuator as shown in FIG. 4A. The gas treatment device was then evaluated. The evaluation results are shown in Table 1.

### Example 7

A gas treatment device was manufactured in the same manner as Example 5 except that first electrodes and second electrodes are formed in two rows as shown in FIGS. 10A (a cross-sectional view in a longitudinal direction), 10B (a view when viewed from a second opening), and 10C (a perspective view). The gas treatment device was then evaluated. The evaluation results are shown in Table 1.

### Comparative Example 1

A gas treatment device was manufactured in the same manner as Example 1 except that an ozone generator was used instead of a plasma actuator as an ozone generation device and air was supplied from a first opening using a fan. The gas treatment device was then evaluated. The evaluation results are shown in Table 1.

In this Comparative Example, air was supplied from the first opening by the fan, causing turbulence in a cylindrical housing, immediate the deactivation of active oxygen, loss of the decolorization effect of methylene blue. As a result, the sterilization effect was significantly reduced.

### Comparative Example 2

The gas treatment device manufactured in Example 1 was prepared. Then, the above “2-1. Confirmation Test for Active Oxygen” and “2-2. Treatment (Sterilization) Test” were conducted in the same manner as Example 1 except that the ultraviolet lamp was not operated. The results are shown in Table 1.

TABLE 1

| | | | | | | | | | Reduction rate | |
|---|---|---|---|---|---|---|---|---|---|---|
| | | | Housing | | | | | | of absorbance | |
| | | Constituting | Cross- | First opening | | Second opening | | | of methylene | Sterilization |
| | | schematic | sectional | | Diameter | | Diameter | | blue | rate |
| Aspect | Example No | view | shape | Shape | (mm) | Shape | (mm) | Dielectric | (%) | (%) |
| 1 | Example 1 | 2A | Circle | Circle | 20 | Circle | 20 | Polyimide sheet | 88 | 98.00 |
| | Example 2 | 2A | Circle | Circle | 20 | Circle | 20 | Silicone resin | 90 | 99.50 |
| | Example 3 | 7A | Rectangle | Rectangle | 20 | Rectangle | 20 | Silicone resin | 90 | 99.50 |
| | Example 4 | 8A | Circle | Circle | 20 | Circle | 20 | Silicone resin | 98 | 100.00 |
| | Example 5 | 9A | Circle | Circle | 20 | Circle | 20 | Polyimide sheet | 85 | 96.50 |
| 2 | Example 6 | 4A | Circle | Circle | 20 | Circle | 20 | Silicone resin | 90 | 99.50 |
| | | | | | | | | (housing) | | |
| | Example 7 | 10A | Circle | Circle | 20 | Circle | 20 | Silicone resin | 98 | 100.00 |
| | | | | | | | | (housing) | | |
| 1 | Comparative | 2A | Circle | Circle | 20 | Circle | 20 | — | 15 | 70.00 |
| | Example 1 | | | | | | | | | |
| 1 | Comparative | 2A | Circle | Circle | 20 | Circle | 20 | Polyimide sheet | 1 | 15.00 |
| | Example 2 | | | | | | | | | |

In the table, Aspect 1 represents the first aspect in which the cylindrical housing includes the plasma actuator, and Aspect 2 represents the second aspect in which the cylindrical housing itself includes the dielectric, and in which the cylindrical housing is used as the dielectric portion of the plasma actuator.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A gas treatment device comprising:
a cylindrical housing having a first opening and a second opening on a side opposite to the first opening;
a plasma actuator arranged inside of the housing; and
an ozone decomposition device, wherein
the plasma actuator comprises a first electrode, a dielectric, and a second electrode,
the dielectric is interposed between the first electrode and the second electrode,
the first electrode and the second electrode are electrically insulated from each other,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the plasma actuator is arranged so that the first direction representing a blowing-out direction of the induced flow is oriented toward the second opening, and causes an airflow containing gas flowing in from the first opening and oriented from the first opening toward the second opening to be generated inside of the housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

2. The gas treatment device according to claim 1, wherein
the plasma actuator and the ozone decomposition device are arranged so that the airflow containing the active oxygen flows to an outside of the gas treatment device from the second opening, and
gas as an object to be treated, which is present in an outflow direction of the airflow containing the active oxygen, is treated by the airflow containing the active oxygen flowing to the outside of the gas treatment device from the second opening.

3. The gas treatment device according to claim 2, wherein
the plasma actuator is arranged over an entire circumference in a peripheral direction inside of the housing.

4. The gas treatment device according to claim 1, wherein
the plasma actuator is arranged at a plurality of places between the first opening and the second opening of the housing.

5. The gas treatment device according to claim 1, wherein
the dielectric is a silicone resin.

6. The gas treatment device according to claim 1, wherein
the cylindrical housing has a cylindrical shape or a rectangular cylindrical shape.

7. The gas treatment device according to claim 1, wherein
the ozone decomposition device is at least one device selected from a group consisting of
an ultraviolet light source irradiating the airflow with ultraviolet rays to generate the active oxygen in the airflow,
a heating device heating the airflow to generate the active oxygen in the airflow, and
a humidifying device humidifying the airflow to generate the active oxygen in the airflow.

8. The gas treatment device according to claim 1, wherein
the ozone decomposition device comprises at least an ultraviolet light source irradiating the airflow with ultraviolet rays to generate the active oxygen in the airflow.

9. A gas treatment device comprising:
a cylindrical housing having a first opening and a second opening on a side opposite to the first opening; and
an ozone decomposition device, wherein
the cylindrical housing comprises a dielectric,
a first electrode representing an exposed electrode provided to coat a part of an inner surface is arranged on the inner surface of the cylindrical housing, and a second electrode electrically insulated from the first electrode via the dielectric is arranged outside of the inner surface of the housing in a cross section of the cylindrical housing along an axial direction,
when a voltage is applied between the first electrode and the second electrode, the gas treatment device generates a dielectric barrier discharge oriented from the first electrode toward the second electrode, and blows out an induced flow containing ozone toward a direction of the second opening representing one direction along the inner surface of the housing from the first electrode, and
the gas treatment device causes an airflow containing gas flowing in from the first opening and oriented from the first opening toward the second opening to be generated inside of the cylindrical housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

10. The gas treatment device according to claim 9, wherein
the first electrode, the second electrode, and the ozone decomposition device are arranged so that the airflow containing the active oxygen flows to an outside of the gas treatment device from the second opening, and
gas as an object to be treated, which is present in an outflow direction of the airflow containing the active oxygen, is treated by the airflow containing the active oxygen flowing to the outside of the gas treatment device from the second opening.

11. The gas treatment device according to claim 9, wherein
the dielectric is a silicone resin.

12. The gas treatment device according to claim 9, wherein
the cylindrical housing has a cylindrical shape or a rectangular cylindrical shape.

13. The gas treatment device according to claim 9, wherein
the ozone decomposition device is at least one device selected from a group consisting of
an ultraviolet light source irradiating the airflow with ultraviolet rays to generate the active oxygen in the airflow,
a heating device heating the airflow to generate the active oxygen in the airflow, and
a humidifying device humidifying the airflow to generate the active oxygen in the airflow.

14. The gas treatment device according to claim 9, wherein
the ozone decomposition device comprises at least an ultraviolet light source irradiating the airflow with ultraviolet rays to generate the active oxygen in the airflow.

15. A gas treatment method using active oxygen, the method comprising:
a step of preparing a gas treatment device, wherein
the gas treatment device comprises
a cylindrical housing having a first opening and a second opening on a side opposite to the first opening,
a plasma actuator arranged inside of the housing, and
an ozone decomposition device,
the plasma actuator comprises a first electrode, a dielectric, and a second electrode,
the dielectric is interposed between the first electrode and the second electrode,
the first electrode and the second electrode are electrically insulated from each other,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the plasma actuator is arranged so that the first direction representing a blowing-out direction of the induced flow is oriented toward the second opening, and causes an airflow containing gas flowing in from the first opening and oriented from the first opening toward the second opening to be generated inside of the housing by the induced flow,
the ozone decomposition device decomposes the ozone contained in the airflow to generate active oxygen in the airflow, and the induced flow results in an airflow containing the active oxygen, and
the gas flowing in from the first opening is treated by the active oxygen.

16. The gas treatment method according to claim 15, wherein
the plasma actuator and the ozone decomposition device are arranged so that the airflow containing the active oxygen flows to an outside of the gas treatment device from the second opening, and
gas as an object to be treated, which is present in an outflow direction of the airflow containing the active oxygen, is treated by the airflow containing the active oxygen flowing to the outside of the gas treatment device from the second opening.

17. The gas treatment method according to claim 15, wherein
the ozone decomposition device is at least one device selected from a group consisting of
an ultraviolet light source irradiating the airflow with ultraviolet rays to generate the active oxygen in the airflow,
a heating device heating the airflow to generate the active oxygen in the airflow, and
a humidifying device humidifying the airflow to generate the active oxygen in the airflow.

18. The gas treatment method according to claim 15, wherein
the ozone decomposition device comprises at least an ultraviolet light source irradiating the airflow with ultraviolet rays to generate the active oxygen in the airflow.
